(19) United States
(12) Patent Application Publication
(10) Pub. No.: US 2018/0228422 A1
(43) Pub. Date: Aug. 16, 2018

(54) METHODS AND SYSTEMS FOR EVALUATING AGE-RELATED MEMORY LOSS

(71) Applicant: THE TRUSTEES OF COLUMBIA UNIVERSITY IN THE CITY OF NEW YORK, New York, NY (US)

(72) Inventors: Adam M. BRICKMAN, Ardsley, NY (US); Scott A. SMALL, Millerton, NY (US); Lok-Kin YEUNG, New York, NY (US)

(73) Assignee: THE TRUSTEES OF COLUMBIA UNIVERSITY IN THE CITY OF NEW YORK, New York, NY (US)

(21) Appl. No.: 15/506,990

(22) PCT Filed: Sep. 15, 2015
(86) PCT No.: PCT/US15/50158

§ 371 Date: Feb. 27, 2017

Related U.S. Application Data
(60) Provisional application No. 62/050,503, filed on Sep. 15, 2014.

(51) Int. Cl. A61B 5/00; G06F 3/0484; G06F 17/30

(52) U.S. Cl. CPC A61B 5/4064; A61B 5/7435; A61B 5/162; G06F 17/30244; G06F 3/04842

(57) ABSTRACT

Methods and systems for measuring brain function of a person are disclosed. In some embodiments, the methods include the following: providing a database including digital image files that define intersecting sinusoidal functions; conducting matching trials including displaying a first image selected from the digital image files for a first amount of time, displaying no images for a second amount of time, and displaying both the first image and a second image, prompting the person to identify the first image, recording whether the person correctly identified the first image, and recording an amount of time to complete the matching trial; and conducting recognition trials including displaying an image selected from the digital image files, prompting the person to identify whether the image was displayed in the matching trials, recording whether the person correctly identified whether the image was displayed, and recording an amount of time to complete the recognition trial.

### CROSS REFERENCE TO RELATED APPLICATION(S)

This application is the National Stage filing of International Patent Application PCT/US2015/050158, filed Sep. 15, 2015, which claims the benefit of U.S. Provisional Application No. 62/050,503, filed Sep. 15, 2014, each of which is incorporated by reference as if disclosed herein in its entirety.

### BACKGROUND

Nearly all studies investigating the effect of aging and disease on human cognition employ a battery of neuropsychological tests that have been characterized and standardized through decades of experience. Typically, these batteries include memory tests putatively sensitive to the function of the hippocampal formation in general. Without wishing to be bound by theory, each region of the hippocampal circuit plays a distinct cognitive operation. Nevertheless, the effect of aging on hippocampal function is often confounded by disease, in particular AD, which commonly occurs in the context of aging and can cause hippocampal dysfunction independent of age. When attempting to isolate the hippocampal pattern of dysfunction reflective of aging per se, it is therefore important to exclude the effects of AD. It has been confirmed that EC function is associated with the amount of retained information over a brief delay on declarative memory tasks, which are also among the most sensitive cognitive measures in early AD. Therefore an optimized cognitive task, e.g., test or process that requires use of a particular part of a person's brain, which overlaps with the anatomical site of age-related DG dysfunction and is correlated selectively with the precise site of DG function and normal aging was desired.

Human fMRI studies have established that the DG, not the EC, plays an important role in pattern separation. Pattern separation is the computational process by which the hippocampal formation orthogonalizes the neural representation of similar stimuli. In addition to various neuropsychological tests designed to evaluate the global hippocampal circuit, the Benton Visual Retention Test (BVRT) localized cognitive activity to the DG among healthy subjects of advanced age. During administration of the BVRT, a subject is shown a series of designs and then depending on the variation of the asked to reproduce the design they saw after 10 seconds, after 5 seconds, the subject is asked to recreate the design while being allowed to view the design, the subject is asked to recreate the design after not looking at it for a period of 15 seconds, the subject views the design and then must select the design from a group of four similar design. Despite being developed over 50 years ago and being ecologically validated, however, the BVRT has a number of practical limitations that are problematic for an intervention study. Firstly, the test items on the BVRT are not sufficiently challenging for healthy subjects, leading to a “ceiling effect” and a non-normal performance distribution. Secondly, because there are only a limited number of test items, the BVRT is ill-suited for a repeated-measures design,

### SUMMARY

Systems and methods according to the disclosed subject matter include tests for evaluating memory age-related memory loss. Systems and methods according to the present disclosure overcome the limitations of the Benton Visual Retention Test (BVRT) and improve the practical utility to provide a hippocampal-dependent memory task that is particularly sensitive to an aging DG.

Systems and methods according to the disclosed subject matter were designed using the observation that the DG is particularly engaged in the pattern separation of visually similar object, e.g., by following a “novel object recognition” procedure, which has been shown to localize to the hippocampal circuit, and more particularly the DG.

Systems and methods according to the disclosed subject matter include displaying particular images to test subjects for various periods of time and determining whether the subject is able to identify a particular image and the associated response time of the subject.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic diagram of methods and systems according to some embodiments of the disclosed subject matter;

FIG. 2 is a chart of a method according to some embodiments of the disclosed subject matter;

FIG. 3 is a schematic diagram of methods and systems according to some embodiments of the disclosed subject matter;

FIG. 4 is a chart showing the normal distribution of performance by healthy individuals taking tests according to methods and systems of the disclosed subject matter;

FIG. 5 is a chart showing a general trend of decreased performance with an increase in age of the subject in assessments developed according to methods and systems of the disclosed subject matter;

FIG. 6 is a three dimensional rendering of the bilateral hippocampal circuit (6A) derived from a group-wise template of multiple axial slices (6B), generated using the native sub-millimeter resolution of cerebral blood volume (CBV) maps; and

FIGS. 7 and 8 are a voxel-based analysis that shows the decline in CBV in the body of the hippocampal circuit (7A and 8A) and a scatter plot (7B and 8B) showing the association between age and mean CBV from all significantly correlated voxels.

### DETAILED DESCRIPTION

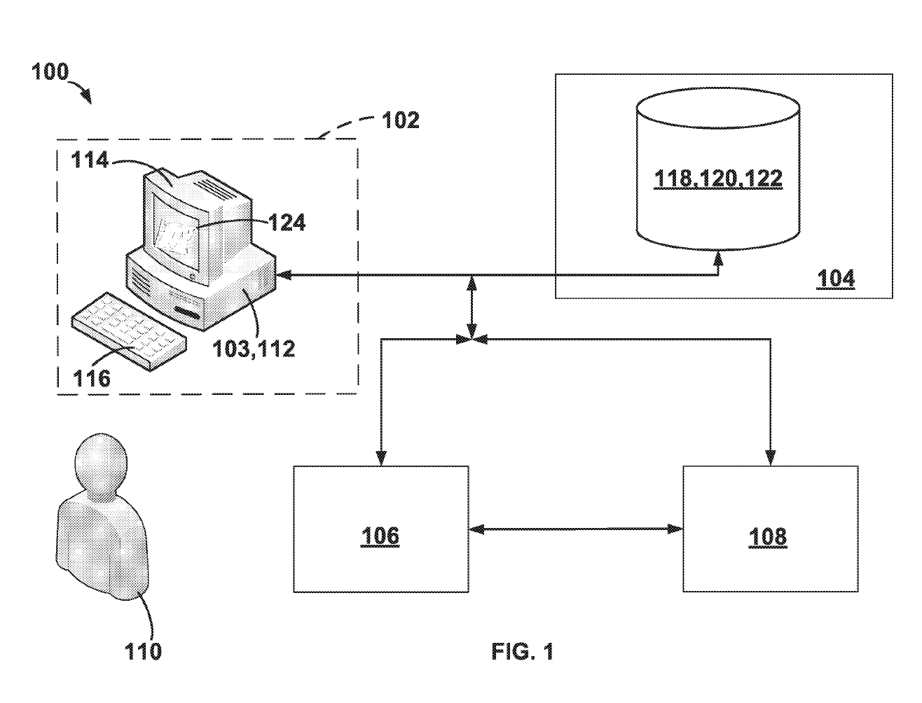

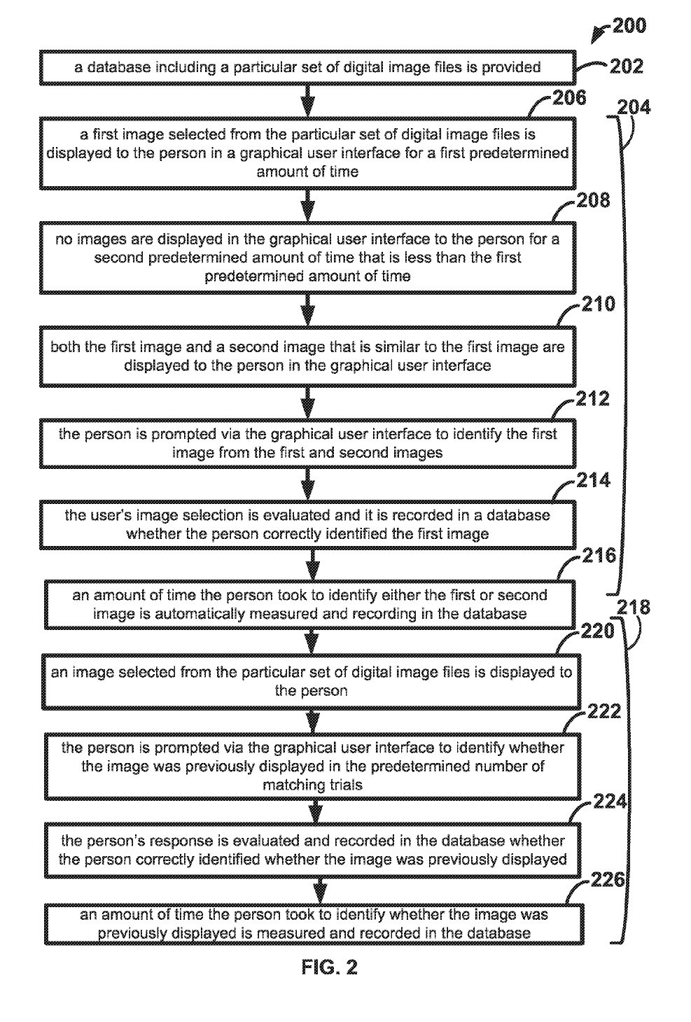

Referring now to FIGS. 1 and 2, aspects of the disclosed subject matter include systems and methods for measuring brain function and evaluating age-related memory loss of a person. As shown in FIG. 1, some embodiments include a system 100 having a computer module 102 including a microprocessor 103, an image generation module 104, a matching trial module 106, and a recognition trial module 108. Image generation module 104, matching trial module 106, and recognition trial module 108 are defined by a set of instructions executed by microprocessor 103 under direction of a person 110.

Computer module 102 includes microprocessor 103, a computer readable medium 112, a graphical user interface 114, and an input device 116, e.g., keyboard and/or mouse, all of which are interconnected. In some embodiments, graphical user interface 114 is touch sensitive, allowing a person to interact with software and images displayed. In some embodiments, computer module 102 includes a laptop computer, a tablet device, and/or other known computer devices.

Image generation module 104 includes a database 118 in digital communication with computer module 102. Database 118 includes a particular set of digital image files 120. Each of particular set of digital image files 120 includes data 122 that defines intersecting sinusoidal functions 124. In some embodiments, each of particular set of digital image files 120 is a particular closed-loop Lissajous figure (see example in FIG. 1) defined by the following functions:

x(t)=sin(a(t)+d); (1)

y(t)=sin(b(t)). (2)

In some embodiments, for functions (1) and (2), if a is equal to one of 1, 2, 3, 4, 5, 6, 7, 8, 11, b is equal to one of 1, 2, 3, 4, 5, 6, and b is not divisible by a. In some embodiments, for functions (1) and (2), if a equals 8, b is equal to one of 2, 3, 4, 5, 6, 7, and if a equals 11, b is equal to one of 2, 3, 4, 5, 6, 7, 8, 9, 10. In some embodiments, for functions (1) and (2), d is equal to one of 1, 2, 3, 4, 5.

Matching trial module 106 includes executable instructions for conducting a predetermined number of matching trials. Each of the predetermined number of matching trials includes the following: (1) displaying to person 110 in graphical user interface 114 a first image selected from particular set of digital image files 120 for a first predetermined amount of time; (2) displaying no images in the graphical user interface to the person for a second predetermined amount of time that is less than the first predetermined amount of time; (3) displaying both the first image and a second image that is similar to the first image to the person in the graphical user interface; (4) prompting the person via the graphical user interface to identify the first image from the first and second images using input device 116; (5) evaluating and recording in database 118 whether the person correctly identified the first image; and (6) automatically measuring and recording in the database an amount of time the person took to identify either the first or second image.

Recognition trial module 108 includes executable instructions for conducting a predetermined number of recognition trials. Each of the predetermined recognition trials including the following: (1) displaying to person 110 an image selected from particular set of digital image files 120; (2) prompting the person via graphical user interface 114 to identify whether the image was previously displayed in the predetermined number of matching trials using input device 116; (3) evaluating and recording in database 118 whether the person correctly identified whether the image was previously displayed; and (4) automatically measuring and recording in the database an amount of time the person took to identify whether the image was previously displayed. In some embodiments, the images displayed during predetermined number of recognition trials and not displayed during the predetermined number of matching trials have the same approximate value of d.

Referring now to FIG. 2, some embodiments include a method 200 of measuring brain function of a person. At 202, a database including a particular set of digital image files is provided. Each of the particular set of digital image files includes data that defines intersecting sinusoidal functions. In some embodiments, there is no particular set of digital image files. Rather, digital image files are automatically generated according to predetermined functions and predetermined parameters, e.g., as discussed with respect to system 100 above, and then displayed to a person.

At 204, a predetermined number of matching trials are conducted, each of which includes steps 206-216. At 206, a first image selected from the particular set of digital image files is displayed to the person in a graphical user interface for a first predetermined amount of time. At 208, no images are displayed in the graphical user interface to the person for a second predetermined amount of time that is less than the first predetermined amount of time. At 210, both the first image and a second image that is similar to the first image are displayed to the person in the graphical user interface. At 212, the person is prompted via the graphical user interface to identify the first image from the first and second images. At 214, the user's image selection is evaluated and it is recorded in a database whether the person correctly identified the first image. At 216, an amount of time the person took to identify either the first or second image is automatically measured and recording in the database.

At 218, a predetermined number of recognition trials are conducted, each of which includes steps 220-226. At 220, an image selected from the particular set of digital image files is displayed to the person. In some embodiments, the images displayed in the predetermined number of recognition trials include an equal number of target images and foil images. The target images are images that were displayed in the predetermined number of matching trials and the foil images are images that were not displayed in the predetermined number of matching trials. At 222, the person is prompted via the graphical user interface to identify whether the image was previously displayed in the predetermined number of matching trials. At 224, the person's response is evaluated and it is recorded in the database whether the person correctly identified whether the image was previously displayed. At 226, an amount of time the person took to identify whether the image was previously displayed is automatically measured and recorded in the database.

In some embodiments, the following various data are collected and evaluated: the mean reaction time, e.g., measured in milliseconds, for correct rejections of foil images during the recognition trials; the number correct; percent correct; other mean reaction times from correct non-foil image and/or non-recognition trials and incorrect non-foil image and/or non-recognition trials; number of hits; number of misses; number of false alarms; etc.

Some embodiments of the disclosed subject matter are directed to a system for administering tests according to the disclosed methods and recording the results. In some embodiments, the system comprises a series of software applications to handle the various duties of task administration and result recordation. In some embodiments, the software is loaded onto a computer readable medium. In some embodiments the computer readable medium includes a database. In some embodiments the database includes the set of images for use in the test, as well as software for presenting the images to a subject consistent with the method parameters. In some embodiments, the images are presented to the subject on a digital screen, such as that of a desktop, laptop, or tablet computer or smart phone.

As mentioned above, in some embodiments, the screen is touch sensitive, allowing the subject to interact with the software and the images displayed thereon. In some embodiments the subject is given a separate input device to interact with the software and images used. In some embodiments, software executing on the computer readable medium is configured to measure the reaction time of a subject when the subject correctly identifies a foil image in the recognition test.

In some embodiments, the database also includes prior subject test results from a broad range of ages. In some embodiments, the system includes software capable to comparing a subject's performance to past performance of similarly aged subjects to diagnose possible age-related memory decline.

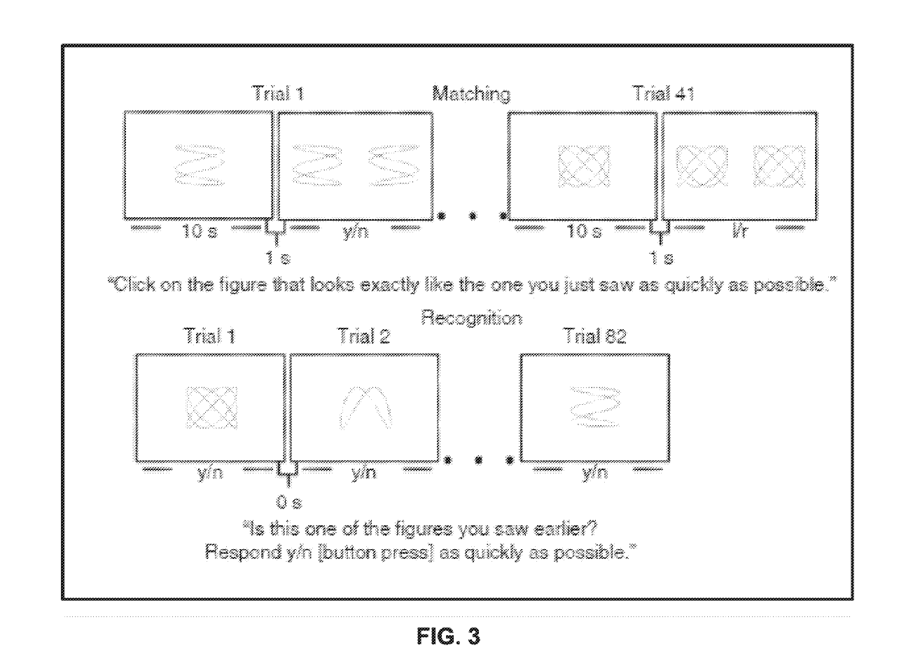

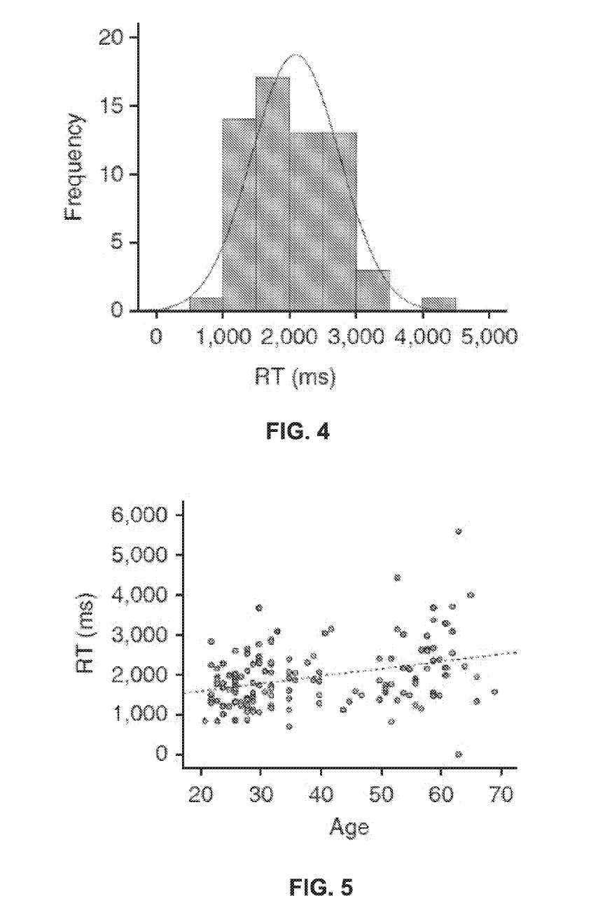

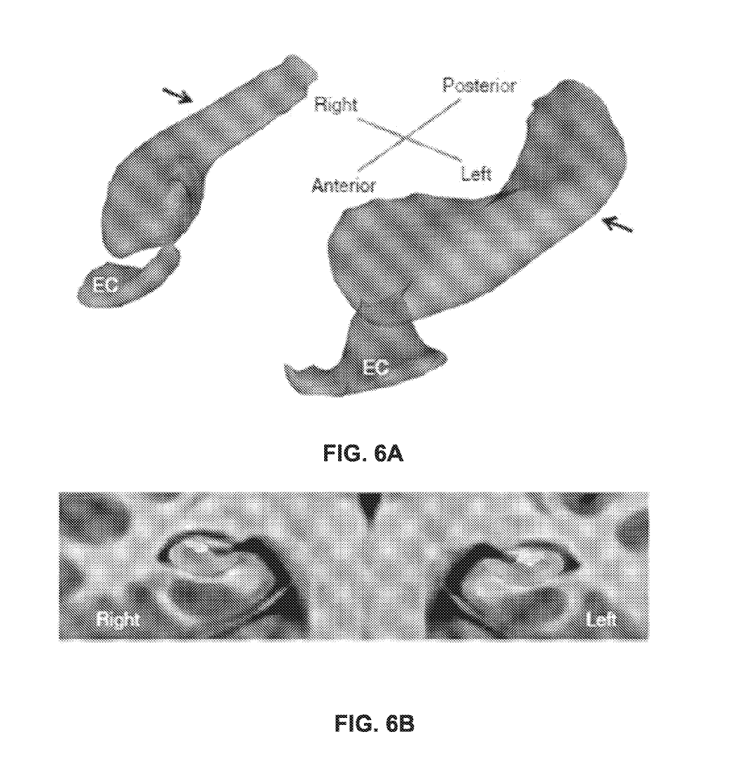

Methods and systems according to the disclosed subject matter were tested and validated for their effectiveness in evaluating age-related memory loss in an experiment with 62 younger subjects with a mean age of 21.12±0.70 years. An exemplary flow chart of both the matching and recognition trials can be found at FIG. 3. As shown in FIG. 4, all subjects were able to understand the instructions and performance was normally distributed (Kolmogorov-Smirnov Test Statistic=0.091, p=0.20), Twelve participants were tested following a 3-month interval; the ICC was 0.743, which indicates an acceptable test re-test reliability for the scale. Methods and systems according to the disclosed subject matter were then administered to 149 healthy subjects of a more advanced age, ranging from 21-69 years of age. As shown in FIG. 5, it was discovered that performance on the showed an age-related worsening. Specifically, the mean reaction time for correct rejections of foil images during recognition trial increased at a rate of approximately 220 ms per decade (β=22.31, p<0.001).

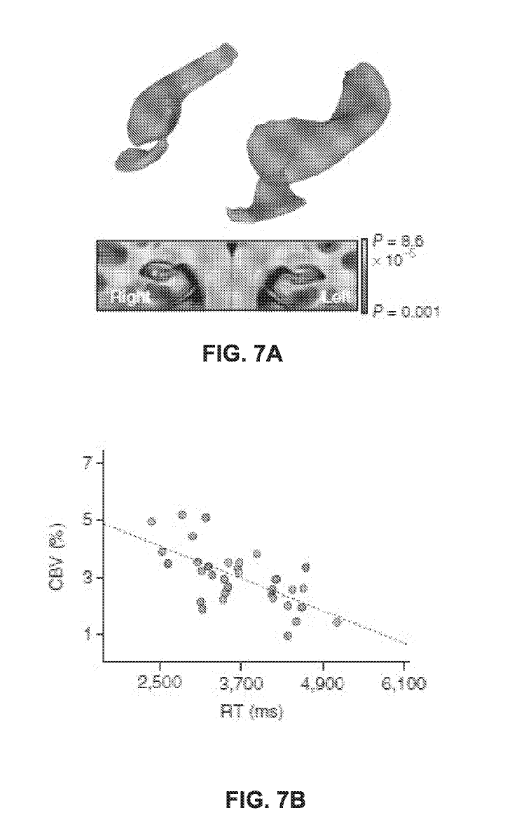

To map the precise localization of age-related changes within the hippocampal circuit, this processing approach was applied to CBV scans acquired from 35 of the healthy individuals, ranging from 21-65 years of age, and generated a 3-dimensional rendering from the groupwise template. As shown in FIG. 6, the approach generated a high fidelity rendering of the bilateral hippocampal circuit throughout its long axis, while the sub-millimeter resolution of the native CBV scans preserved the regional anatomy within its transverse axis. Such a rendering allows the results of the voxel-based analyses to be displayed in a single snapshot of the hippocampal circuit.

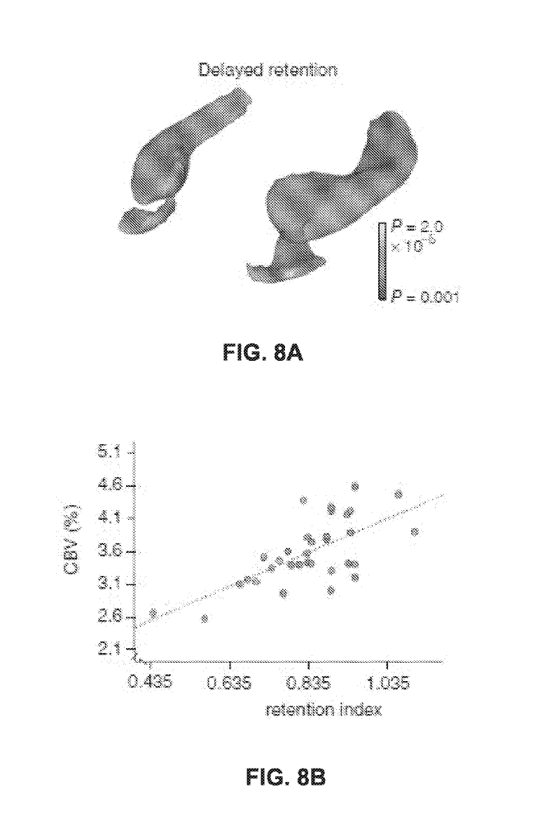

As shown in FIG. 7, better performance correlated selectively with higher DG CBV in the body of the hippocampus, right greater than left. A voxel based analysis of the 35 healthy individuals shows that better performance on the task correlated with greater CBV in the body of the hippocampal circuit (7A—left upper panel), right greater than left, in the dentate gyrus (7A—left lower panel; coronal section, color coded by degree of significance). A scatter plot (8B) shows the association between task performance and single-subject mean CBV from all significantly correlated voxels (β=−0.633, r2=0.465 p<0.001). A delayed retention task was also administered to the 35 subjects, because previous studies suggest that this cognitive operation localizes to the EC. The delayed retention task was a modified version of the Rey Auditory Verbal Learning Test, a list learning and declarative memory task, and the amount of retention of information over a brief interval was calculated. As shown in FIG. 8, confirming previous observations, performance on delayed retention was found to correlate selectively with EC CBV, thereby establishing an anatomical double dissociation with the task. A voxel based analysis within the same group showed that better performance on a delayed retention task correlated with greater CBV in the entorhinal cortex (8A). A scatter plot (8B) shows the association between performance on this delayed retention task and single-subject mean CBV from all significantly correlated voxels (β=0.693, r2=0.562 p<0.001).

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

1. A method of measuring brain function of a person comprising:
providing a database including a particular set of digital image files, each of said particular set of digital image files including data that defines intersecting sinusoidal functions;
conducting a predetermined number of matching trials, each of said predetermined number of matching trials including displaying to said person in a graphical user interface a first image selected from said particular set of digital image files for a first predetermined amount of time, and displaying both said first image and a second image that is similar to said first image to said person in said graphical user interface, prompting said person to identify said first image from said first and second images, evaluating and recording in said database whether said person correctly identified said first image, and automatically measuring and recording in said database an amount of time said person took to identify either said first or second image; and
conducting a predetermined number of recognition trials, each of said predetermined recognition trials including displaying to said person an image selected from said particular set of digital image files, prompting said person to identify whether said image was previously displayed in said predetermined number of matching trials, evaluating and recording in said database whether said person correctly identified whether said image was previously displayed, and automatically measuring and recording in said database an amount of time said person took to identify whether said image was previously displayed.

2. The method according to claim 1, wherein each of said particular set of digital image files is a particular closed-loop Lissajous figure defined by x(t)=sin(a(t)+d) and y(t)=sin(b(t)).

3. The method according to claim 2, wherein a is equal to one of 1, 2, 3, 4, 5, 6, 7, 8, 11, b is equal to one of 1, 2, 3, 4, 5, 6, and b is not divisible by a.

4. The method according to claim 3, wherein if a equals 8, b is equal to one of 2, 3, 4, 5, 6, 7, and if a equals 11, b is equal to one of 2, 3, 4, 5, 6, 7, 8, 9, 10.

5. The method according to claim 4, wherein d is equal to one of 1, 2, 3, 4, 5.

6. The method according to claim 5, wherein said images displayed during said predetermined number of recognition trials and not displayed during said predetermined number of matching trials have the same approximate value of d.

7. The method according to claim 2, wherein said images displayed in said predetermined number of recognition trials include an equal number of target images and foil images, said target images being images that were displayed in said predetermined number of matching trials and said foil images being images that were not displayed in said predetermined number of matching trials.

8. A system for measuring brain function of a person comprising:
a computer module having interconnected components, said components including a microprocessor, a computer readable medium, a graphical user interface, and an input device;
an image generation module including a database having a particular set of digital image files, each of said particular set of digital image files including data that defines intersecting sinusoidal functions, said database being in digital communication with said computer module;
a matching trial module for conducting a predetermined number of matching trials, each of said predetermined number of matching trials including displaying to said person in said graphical user interface a first image selected from said particular set of digital image files for a first predetermined amount of time, and displaying both said first image and a second image that is similar to said first image to said person in said graphical user interface, prompting said person to identify said first image from said first and second images using said input device,
evaluating and recording in said database whether said person correctly identified said first image, and automatically measuring and recording in said database an amount of time said person took to identify either said first or second image; and
a recognition trial module for conducting a predetermined number of recognition trials, each of said predetermined recognition trials including displaying to said person an image selected from said particular set of digital image files, prompting said person to identify whether said image was previously displayed in said predetermined number of matching trials using said input device, evaluating and recording in said database whether said person correctly identified whether said image was previously displayed, and automatically measuring and recording in said database an amount of time said person took to identify whether said image was previously displayed;
wherein said image generation module, said matching trial module, and said recognition trial module are defined by a set of instructions executed by said microprocessor under direction of said person.

9. The system according to claim 8, wherein each of said particular set of digital image files is a particular closed-loop Lissajous figure defined by x(t)=sin(a(t)+d) and y(t)=sin(b(t)).

10. The system according to claim 9, wherein a is equal to one of 1, 2, 3, 4, 5, 6, 7, 8, 11, b is equal to one of 1, 2, 3, 4, 5, 6, and b is not divisible by a.

11. The system according to claim 10, wherein if a equals 8, b is equal to one of 2, 3, 4, 5, 6, 7, and if a equals 11, b is equal to one of 2, 3, 4, 5, 6, 7, 8, 9, 10.

12. The system according to claim 11, wherein d is equal to one of 1, 2, 3, 4, 5.

13. The system according to claim 12, wherein said images displayed during said predetermined number of recognition trials and not displayed during said predetermined number of matching trials have the same approximate value of d.

14. A method of measuring brain function of a person comprising:
conducting a predetermined number of matching trials, each of said predetermined number of matching trials including automatically generating and displaying to said person in a graphical user interface a particular closed-loop Lissajous figure defined by x(t)=sin(a(t)+d), y(t)=sin(b(t)) for a first predetermined amount of time, and displaying both said first image and generating and displaying a second image that is similar to said first image to said person in said graphical user interface, prompting said person to identify said first image from said first and second images, recording values of a, b, and d for said first and second images in a database, evaluating and recording in said database whether said person correctly identified said first image, and automatically measuring and recording in said database an amount of time said person took to identify either said first or second image; and
conducting a predetermined number of recognition trials, each of said predetermined recognition trials including displaying to said person an image selected from said particular set of digital image files, prompting said person to identify whether said image was previously displayed in said predetermined number of matching trials, evaluating and recording in said database whether said person correctly identified whether said image was previously displayed, and automatically measuring and recording in said database an amount of time said person took to identify whether said image was previously displayed.

15. The method according to claim 14, wherein said first and second images generated and displayed in said predetermined number of matching trials are randomly selected.

16. The method according to claim 14, wherein a is equal to one of 1, 2, 3, 4, 5, 6, 7, 8, 11, b is equal to one of 1, 2, 3, 4, 5, 6, and b is not divisible by a.

17. The method according to claim 16, wherein if a equals 8, b is equal to one of 2, 3, 4, 5, 6, 7, and if a equals 11, b is equal to one of 2, 3, 4, 5, 6, 7, 8, 9, 10.

18. The method according to claim 17, wherein d is equal to one of 1, 2, 3, 4, 5.

19. The method according to claim 18, wherein said images displayed during said predetermined number of recognition trials and not displayed during said predetermined number of matching trials have the same approximate value of d.

20. The method according to claim 14, wherein said images displayed in said predetermined number of recognition trials include an equal number of target images and foil images, said target images being images that were displayed in said predetermined number of matching trials and said foil images being images that were not displayed in said predetermined number of matching trials.
